(19) United States
(12) Patent Application Publication
(10) Pub. No.: US 2021/0205143 A1
(43) Pub. Date: Jul. 8, 2021

(54) Dressing Interface, Systems, And Methods

(71) Applicant: KCI Licensing, Inc., San Antonio, TX (US)

(72) Inventors: Christopher Brian LOCKE, Bournemouth (GB); Benjamin Andrew PRATT, Poole (GB)

(21) Appl. No.: 16/650,781

(22) PCT Filed: Aug. 30, 2018
(86) PCT No.: PCT/US2018/048885

§ 371 Date: Mar. 25, 2020

Related U.S. Application Data
(60) Provisional application No. 62/565,786, filed on Sep. 29, 2017.

(51) Int. Cl. A61F 13/02; A61L 15/24

(52) U.S. Cl. CPC A61F 13/0223; A61F 2013/00255; A61L 15/24; A61F 13/0216

(57) ABSTRACT

A dressing interface for a negative-pressure treatment system includes a housing. The housing includes an entry surface having first channels. The dressing interface also includes a primary conduit through the housing and terminating on the entry surface, an ancillary conduit through the housing and terminating on the entry surface, and a base coupled to the housing. The base includes an aperture, and a plurality of stand-offs having rounded surfaces. The stand-offs defines second channels configured to facilitate flow of liquid to the first channels through the aperture.

### RELATED APPLICATION

This application claims the benefit, under 35 U.S.C. § 119(e), of the filing of U.S. Provisional Patent Application Ser. No. 62/565,786, entitled “DRESSING INTERFACE, SYSTEMS, AND METHODS,” filed Sep. 29, 2017, which is incorporated herein by reference for all purposes.

### TECHNICAL FIELD

The invention set forth in the appended claims relates generally to tissue treatment systems and more particularly, but without limitation, to a dressing interface, systems, and methods of reducing tissue damage during treatment of wounds.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

There is also widespread acceptance that cleansing a tissue site can be highly beneficial for new tissue growth. For example, a wound can be washed out with a stream of liquid solution, or a cavity can be washed out using a liquid solution for therapeutic purposes. These practices are commonly referred to as “irrigation” and “lavage” respectively. “Instillation” is another practice that generally refers to a process of slowly introducing fluid to a tissue site and leaving the fluid for a prescribed period of time before removing the fluid. For example, instillation of topical treatment solutions over a wound bed can be combined with negative-pressure therapy to further promote wound healing by loosening soluble contaminants in a wound bed and removing infectious material. As a result, soluble bacterial burden can be decreased, contaminants removed, and the wound cleansed.

While the clinical benefits of negative-pressure therapy and instillation therapy are widely known, improvements to therapy systems, components, and processes may benefit healthcare providers and patients.

### BRIEF SUMMARY

New and useful systems, apparatuses, and methods for treating tissue in a negative-pressure therapy environment are set forth in the appended claims. Illustrative embodiments are also provided to enable a person skilled in the art to make and use the claimed subject matter.

For example, in some embodiments, a dressing interface for a therapy system includes housing having a flange and a plurality of rounded protuberances or stand-offs on the flange, which can provide direct and indirect fluid pathways over the flange for manifolding fluids. The protuberances may vary in size from a central area to an edge, and in some embodiments the size may be varied to present a mixture of patterns and textures with no linear features that could create pressure points or strain on tissue.

More generally, in some embodiments, a dressing interface for a negative-pressure treatment system includes a housing. The housing may include an entry surface having first channels. The dressing interface may also include a primary conduit through the housing and an ancillary conduit through the housing. The primary conduit and the ancillary conduit may each have an end that terminates on the entry surface. Additionally, the dressing interface may also include a base coupled to the housing. The base may include an aperture and a plurality of stand-offs having rounded surfaces. In some embodiments, the stand-offs may define second channels configured to facilitate flow of liquid to the first channels through the aperture.

In some embodiments, the bottom surface is formed of polyvinyl chloride (PVC). The stand-offs may have a hardness of about 60 Shore A and a generally round shape, which can provide a gentle tissue interface area. The stand-offs adjacent the aperture defined in the base may have smaller dimensions than stand-offs adjacent an edge of the base. In some examples, the stand-offs may be distributed in an irregular pattern on the bottom surface or in a regular pattern on the bottom surface. Each of the stand-offs may have the same size or a different size.

In some embodiments, the housing may comprise an entry surface having second channels. The second channels can be configured to direct liquid away from the ancillary conduits.

Alternatively, other example embodiments generally relate to a negative-pressure treatment system. The negative-pressure treatment system may comprise a conduit comprising a primary lumen and a secondary lumen, a negative-pressure source coupled to the primary lumen, and a dressing interface coupled to the conduit. The dressing interface in some examples may comprise a base and a housing, first channels associated with the housing and configured to preference fluid into the primary lumen, an aperture in the base, and a plurality of stand-offs having rounded surfaces defining second channels configured to facilitate flow of fluid to the first channels through the aperture.

Some embodiments relate to a system for applying negative pressure to a tissue site. In some example embodiments, the system comprises a primary lumen having a proximate end and a distal end, a secondary lumen having a proximate end and a distal end, a negative-pressure source coupled to the proximate end of the primary lumen, a pressure sensor coupled to the proximate end of the secondary lumen, and a dressing interface. The dressing interface may comprise a housing having a bottom surface, a primary fluid pathway extending through the housing and fluidly coupled to the primary lumen, a secondary fluid pathway extending through the housing and fluidly coupled to the secondary lumen, and stand-offs extending from the bottom surface. The stand-offs may have rounded or otherwise non-linear surfaces and edges defining second channels. The second channels may be configured to facilitate flow of fluid across the bottom surface to the primary fluid pathway.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a functional block diagram of an example embodiment of a system that can provide negative-pressure treatment in accordance with this specification;

FIG. 2 is a schematic view of example of the system of FIG. 1.

FIG. 3 is a top view of an example dressing interface that may be associated with some embodiments of the system of FIG. 1.

FIG. 4 is an end view of the dressing interface of FIG. 3.

FIG. 5 is a perspective view of the dressing interface of FIG. 3.

FIG. 6 is bottom view of the dressing interface of FIG. 6.

FIG. 7 is a cross-sectional view of the dressing interface of FIG. 3.

FIG. 8 is a detailed view of a recessed region of the dressing interface of FIG. 3.

FIG. 9 is a cross-sectional view of the dressing interface of FIG. 6.

FIG. 10 is an exploded view of another example dressing interface that may be associated with some embodiments of the system of FIG. 1.

### DESCRIPTION OF EXAMPLE EMBODIMENTS

The following description of example embodiments provides information that enables a person skilled in the art to make and use the subject matter set forth in the appended claims, but may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

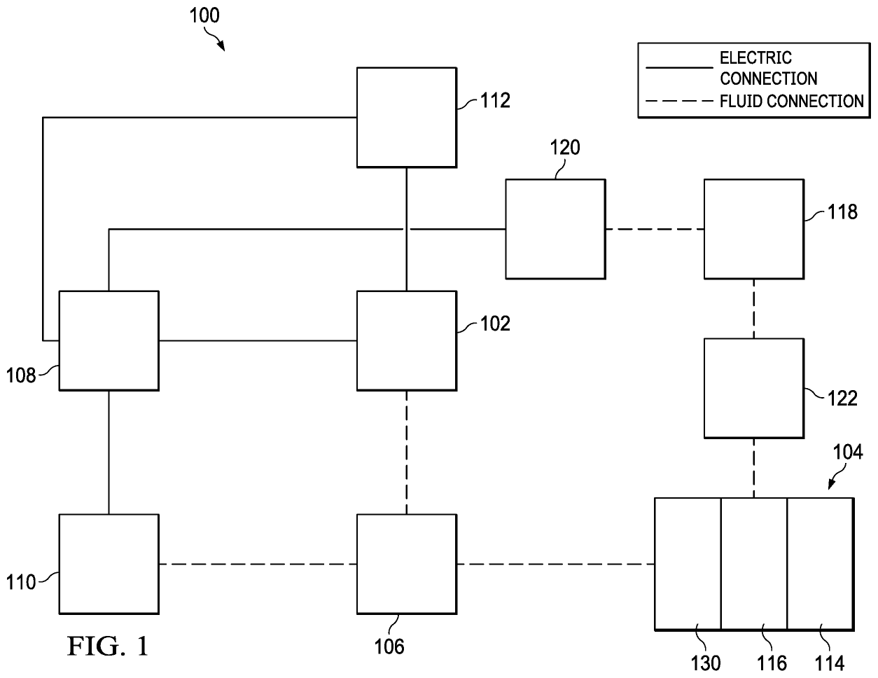

FIG. 1 is a simplified functional block diagram of an example embodiment of a therapy system 100 that can provide negative-pressure therapy to a tissue site in accordance with this specification.

The term “tissue site” in this context broadly refers to a wound, defect, or other treatment target located on or within tissue, including but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

The therapy system 100 may include a source or supply of negative pressure, such as a negative-pressure source 102, a dressing 104, a fluid container, such as a container 106, and a regulator or controller, such as a controller 108, for example. Additionally, the therapy system 100 may include sensors to measure operating parameters and provide feedback signals to the controller 108 indicative of the operating parameters. As illustrated in FIG. 1, for example, the therapy system 100 may include a pressure sensor 110, an electric sensor 112, or both, coupled to the controller 108. As illustrated in the example of FIG. 1, the dressing 104 may comprise or consist essentially of a tissue interface 114, a cover 116, or both in some embodiments.

The therapy system 100 may also include a source of instillation solution. For example, a solution source 118 may be fluidly coupled to the dressing 104, as illustrated in the example embodiment of FIG. 1. The solution source 118 may be fluidly coupled to a positive-pressure source, such as the positive-pressure source 120 in some embodiments, or may be fluidly coupled to the negative-pressure source 102. A regulator, such as an instillation regulator 122, may also be fluidly coupled to the solution source 118 and the dressing 104. In some embodiments, the instillation regulator 122 may also be fluidly coupled to the negative-pressure source 102 through the dressing 104, as illustrated in the example of FIG. 1.

Some components of the therapy system 100 may be housed within or used in conjunction with other components, such as sensors, processing units, alarm indicators, memory, databases, software, display devices, or user interfaces that further facilitate therapy. For example, in some embodiments, the negative-pressure source 102 may be combined with the solution source 118, the controller 108 and other components into a therapy unit.

In general, components of the therapy system 100 may be coupled directly or indirectly. For example, the negative-pressure source 102 may be directly coupled to the container 106, and may be indirectly coupled to the dressing 104 through the container 106. Coupling may include fluid, mechanical, thermal, electrical, or chemical coupling (such as a chemical bond), or some combination of coupling in some contexts. In some embodiments, components may also be coupled by virtue of physical proximity, being integral to a single structure, or being formed from the same piece of material. For example, the negative-pressure source 102 may be electrically coupled to the controller 108. The negative-pressure source 102 may be fluidly coupled to one or more distribution components, which provide a fluid path to a tissue site.

A distribution component is preferably detachable, and may be disposable, reusable, or recyclable. The dressing 104 and the container 106 are illustrative of distribution components. A fluid conductor is another illustrative example of a distribution component. A “fluid conductor,” in this context, broadly includes a tube, pipe, hose, conduit, or other structure with one or more lumina adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, for example, a dressing interface 130 may facilitate coupling a fluid conductor to the dressing 104.

A negative-pressure supply, such as the negative-pressure source 102, may be a reservoir of air at a negative pressure, or may be a manual or electrically-powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure applied to a tissue site may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

The container 106 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

A controller, such as the controller 108, may be a microprocessor or computer programmed to operate one or more components of the therapy system 100, such as the negative-pressure source 102. In some embodiments, for example, the controller 108 may be a microcontroller, which generally comprises an integrated circuit containing a processor core and a memory programmed to directly or indirectly control one or more operating parameters of the therapy system 100. Operating parameters may include the power applied to the negative-pressure source 102, the pressure generated by the negative-pressure source 102, or the pressure distributed to the tissue interface 114, for example. The controller 108 is also preferably configured to receive one or more input signals, such as a feedback signal, and programmed to modify one or more operating parameters based on the input signals.

Sensors, such as the pressure sensor 110 or the electric sensor 112, are generally known in the art as any apparatus configured to detect or measure a physical phenomenon or property, and generally provide a signal indicative of the phenomenon or property that is detected or measured. For example, the pressure sensor 110 and the electric sensor 112 may be configured to measure one or more operating parameters of the therapy system 100. In some embodiments, the pressure sensor 110 may be a transducer configured to measure pressure in a pneumatic pathway and convert the measurement to a signal indicative of the pressure measured. In some embodiments, for example, the pressure sensor 110 may be a piezoresistive strain gauge. The electric sensor 112 may optionally measure operating parameters of the negative-pressure source 102, such as the voltage or current, in some embodiments. Preferably, the signals from the pressure sensor 110 and the electric sensor 112 are suitable as an input signal to the controller 108, but some signal conditioning may be appropriate in some embodiments. For example, the signal may need to be filtered or amplified before it can be processed by the controller 108. Typically, the signal is an electrical signal, but may be represented in other forms, such as an optical signal.

The tissue interface 114 can be generally adapted to contact a tissue site. The tissue interface 114 may be partially or fully in contact with the tissue site. If the tissue site is a wound, for example, the tissue interface 114 may partially or completely fill the wound, or may be placed over the wound. The tissue interface 114 may take many forms, and may have many sizes, shapes, or thicknesses depending on a variety of factors, such as the type of treatment being implemented or the nature and size of a tissue site. For example, the size and shape of the tissue interface 114 may be adapted to the contours of deep and irregular shaped tissue sites. Moreover, any or all of the surfaces of the tissue interface 114 may have projections or an uneven, course, or jagged profile that can induce strains and stresses on a tissue site, which can promote granulation at the tissue site.

In some embodiments, the tissue interface 114 may be a manifold. A “manifold” in this context generally includes any substance or structure providing a plurality of pathways adapted to collect or distribute fluid across a tissue site under pressure. For example, a manifold may be adapted to receive negative pressure from a source and distribute negative pressure through multiple apertures across a tissue site, which may have the effect of collecting fluid from across a tissue site and drawing the fluid toward the source. In some embodiments, the fluid path may be reversed or a secondary fluid path may be provided to facilitate delivering fluid across a tissue site.

In some illustrative embodiments, the pathways of a manifold may be interconnected to improve distribution or collection of fluids across a tissue site. In some illustrative embodiments, a manifold may be a porous foam material having interconnected cells or pores. For example, open-cell foam, porous tissue collections, and other porous material such as gauze or felted mat generally include pores, edges, and/or walls adapted to form interconnected fluid channels. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

The average pore size of a foam may vary according to needs of a prescribed therapy. For example, in some embodiments, the tissue interface 114 may be a foam having pore sizes in a range of 400-600 microns. The tensile strength of the tissue interface 114 may also vary according to needs of a prescribed therapy. For example, the tensile strength of a foam may be increased for instillation of topical treatment solutions. In one non-limiting example, the tissue interface 114 may be a reticulated polyurethane foam such as GRANUFOAM™ dressing or V.A.C. VERAFLO™ dressing, both available from KCI of San Antonio, Tex.

The tissue interface 114 may be either hydrophobic or hydrophilic. In an example in which the tissue interface 114 may be hydrophilic, the tissue interface 114 may also wick fluid away from a tissue site, while continuing to distribute negative pressure to the tissue site. The wicking properties of the tissue interface 114 may draw fluid away from a tissue site by capillary flow or other wicking mechanisms. An example of a hydrophilic foam is a polyvinyl alcohol, open-cell foam such as V.A.C. WHITEFOAM™ dressing available from KCI of San Antonio, Tex. Other hydrophilic foams may include those made from polyether. Other foams that may exhibit hydrophilic characteristics include hydrophobic foams that have been treated or coated to provide hydrophilicity.

The tissue interface 114 may further promote granulation at a tissue site when pressure within the sealed therapeutic environment is reduced. For example, any or all of the surfaces of the tissue interface 114 may have an uneven, coarse, or jagged profile that can induce microstrains and stresses at a tissue site if negative pressure is applied through the tissue interface 114.

In some embodiments, the tissue interface 114 may be constructed from bioresorbable materials. Suitable bioresorbable materials may include, without limitation, a polymeric blend of polylactic acid (PLA) and polyglycolic acid (PGA). The polymeric blend may also include without limitation polycarbonates, polyfumarates, and capralactones. The tissue interface 114 may further serve as a scaffold for new cell-growth, or a scaffold material may be used in conjunction with the tissue interface 114 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the cover 116 may provide a bacterial barrier and protection from physical trauma. The cover 116 may also be constructed from a material that can reduce evaporative losses and provide a fluid seal between two components or two environments, such as between a therapeutic environment and a local external environment. The cover 116 may be, for example, an elastomeric film or membrane that can provide a seal adequate to maintain a negative pressure at a tissue site for a given negative-pressure source. The cover 116 may have a high moisture-vapor transmission rate (MVTR) in some applications. For example, the MVTR may be at least 300 g/m\hat{ }2 per twenty-four hours in some embodiments. In some example embodiments, the cover 116 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained.

An attachment device may be used to attach the cover 116 to an attachment surface, such as undamaged epidermis, a gasket, or another cover. The attachment device may take many forms. For example, an attachment device may be a medically-acceptable, pressure-sensitive adhesive that extends about a periphery, a portion, or an entire sealing member. In some embodiments, for example, some or all of the cover 116 may be coated with an acrylic adhesive having a coating weight between 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

The solution source 118 may also be representative of a container, canister, pouch, bag, or other storage component, which can provide a solution for instillation therapy. Compositions of solutions may vary according to a prescribed therapy, but examples of solutions that may be suitable for some prescriptions include hypochlorite-based solutions, silver nitrate (0.5%), sulfur-based solutions, biguanides, cationic solutions, and isotonic solutions.

The fluid mechanics of using a negative-pressure source to reduce pressure in another component or location, such as within a sealed therapeutic environment, can be mathematically complex. However, the basic principles of fluid mechanics applicable to negative-pressure therapy and instillation are generally well-known to those skilled in the art, and the process of reducing pressure may be described illustratively herein as “delivering,” “distributing,” or “generating” negative pressure, for example.

In general, exudates and other fluids flow toward lower pressure along a fluid path. Thus, the term “downstream” typically implies something in a fluid path relatively closer to a source of negative pressure or further away from a source of positive pressure. Conversely, the term “upstream” implies something relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications (such as by substituting a positive-pressure source for a negative-pressure source) and this descriptive convention should not be construed as a limiting convention.

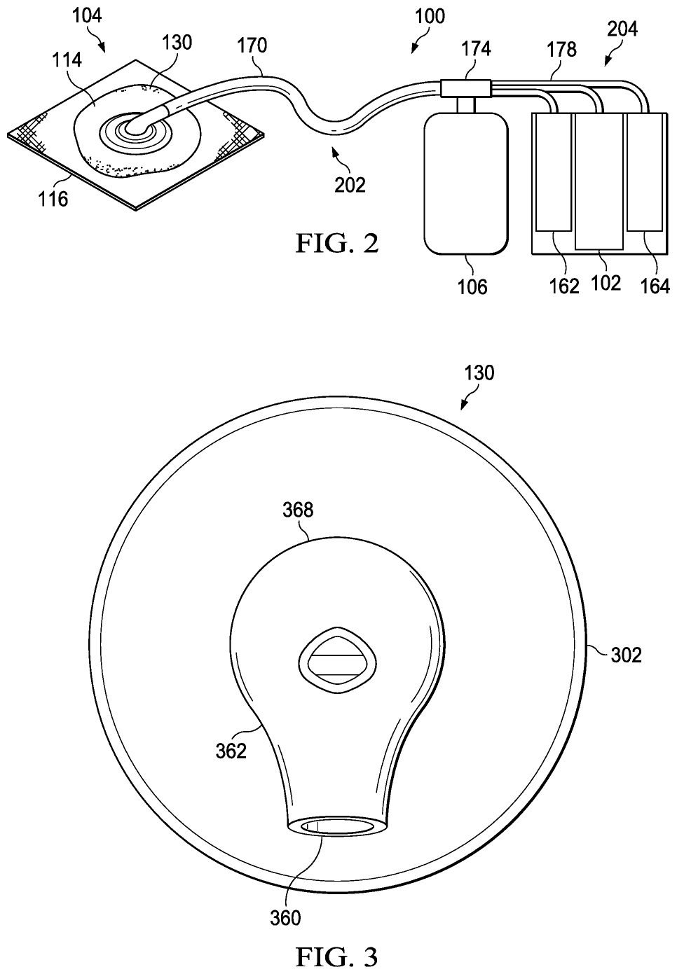

FIG. 2 is a schematic view of an example of the therapy system 100, illustrating additional details that may be associated with some embodiments. In the example embodiment of FIG. 2, the therapy system 100 generally includes the dressing 104, a delivery tube 202, and a therapy unit 204.

As shown in the example of FIG. 2, the dressing interface 130 may be adhered to the cover 116 in some embodiments. For example, the dressing interface 130 may be attached to the cover 116 by an adhesive positioned on the dressing interface 130, the cover 116, or a separate adhesive drape associated with dressing interface 130.

In some examples, the delivery tube 202 may be a fluid conductor including a plurality of fluid pathways therethrough, such as a multi-lumen tube. The delivery tube 202 may comprise one or more tubing sections 170 which, as an assembled structure, can provide a continuous fluid pathway between the dressing interface 130 and a container connector 174. In the example of FIG. 2, the container connector 174 connects the tubing sections 170 to the container 106.

Sections of additional tubing in the form of component tubing 178 likewise may extend from container connector 174 to other components of the therapy unit 204. In certain embodiments, for example, other components may include the negative-pressure source 102 and pressure monitoring components, such as a first pressure monitor 162 and a second pressure monitor 164. Each of the negative-pressure source 102, the first pressure monitor 162, and the second pressure monitor 164 may be individually associated with one of three isolated conduits (tubes or lumens) that extend from the dressing interface 130.

FIG. 3 is a top view of an example of the dressing interface 130, illustrating additional details that may be associated with some embodiments. In some embodiments, the dressing interface 130 may include a flange or other base, such as a base 302. The dressing interface 130 may also include a conduit housing 362, as illustrated in the example of FIG. 3. The conduit housing 362 of FIG. 3 is generally “elbow” shaped, and has an elbow region 368. In other embodiments, the conduit housing 362 may be configured at any desired angle or may extend perpendicularly from the base 302. The conduit housing 362 may be centrally positioned on the base 302 in some embodiments. In other embodiments, the conduit housing 362 may not be centrally positioned on the base 302.

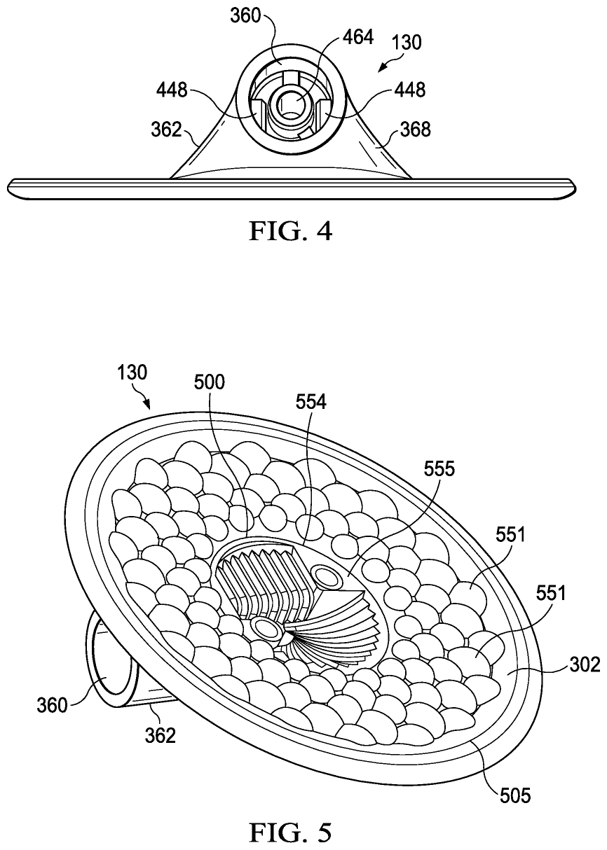

FIG. 4 is an end view of the dressing interface of FIG. 3 illustrating additional details that may be associated with some embodiments. As shown in FIG. 4, the dressing interface 130 may have a low profile construction. In some embodiments, the dressing interface 130 has a height ranging from about 10 mm to about 20 mm (e.g., about 12 mm to about 15 mm). The base 302 defines lateral limits of the dressing interface 130 of FIG. 3. The base 302 may have a diameter ranging from about 30 mm to about 50 mm (e.g., about 35 mm to about 45 mm). For example, a height of about 12.5 mm and a diameter of about 46.0 mm may be suitable for some embodiments.

FIG. 4 further illustrates details that may be associated with the internal configuration of conduit housing 362. As shown, the conduit housing 362 may be configured to receive the tubing sections 170 (shown in FIG. 2). In some embodiments, the conduit housing 362 also defines an aperture 360 therein.

In some embodiments, as shown in the example of FIG. 4, a primary lumen interface 464 may be positioned coaxially within the aperture 360 of the conduit housing 362. One or more ancillary lumen interfaces 448 may also be positioned within the aperture 360 adjacent to the primary lumen interface 464. The primary lumen interface 464 may be centrally located within the conduit housing 362. The ancillary lumen interfaces 448 may be configured to align with corresponding fluid pathways in the tubing sections 170. The tubing sections 170 may be connected to the conduit housing 362 by placing a primary lumen in the tubing sections 170 over the primary lumen interface 464.

FIG. 5 is a perspective view of the dressing interface 130, illustrating additional details that may be associated with some embodiments. In the example of FIG. 5, the base 302 includes an aperture 500, which may be centrally disposed in some embodiments. In some embodiments, the base 302 may include a plurality of protuberances, such as stand-offs 551, formed on a bottom surface of the base 302. The stand-offs 551 may cover at least a portion of the bottom surface of the base 302 between the aperture 500 and an outer edge 505 of the base 302. In some embodiments, the stand-offs 551 can cover substantially all of the bottom surface of the base 302.

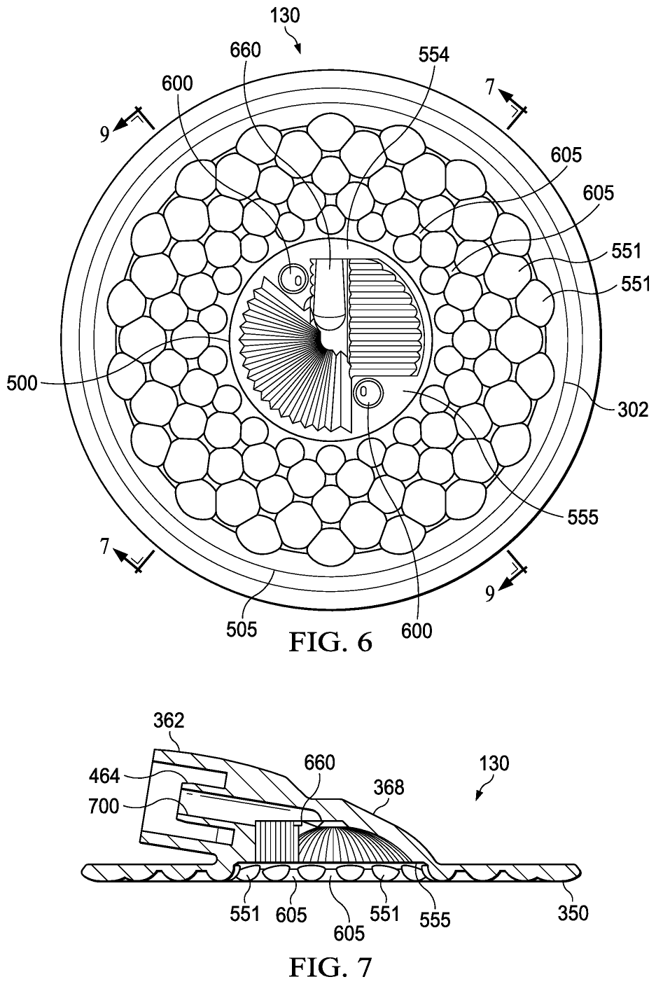

FIG. 6 is bottom view of the dressing interface 130 of FIG. 3, illustrating additional details that may be associated with some embodiments. For example, the base 302 may have channels 605, which may provide direct and indirect fluid pathways to the aperture 500. As illustrated in FIG. 6, the channels 605 may be disposed between the stand-offs 551, and in some examples, may be at least partially defined by adjacent stand-offs 551.

In some embodiments, the stand-offs 551 may be molded with the base 302. The base 302, the stand-offs 551, or both may be formed of a soft material. A material having a hardness of about 60 Shore A may be suitable for some embodiments. For example, the material may be polyvinyl chloride (PVC) or another suitable polymer.

The stand-offs 551 may have rounded or otherwise non-linear surfaces and edges, as illustrated in the example of FIG. 6, such that there are no linear features that may create pressure points and/or strain on tissue. The edges may be curved and/or beveled with no sharp points. The stand-offs 551 may be generally round, oval, and/or hemispherical in shape. In other embodiments, the stand-offs 551 may have other shapes with rounded edges, such as a squircle shape. The stand-offs 551 may be irregularly shaped, have free-form shapes, and/or organic shapes in some embodiments.

The stand-offs 551 may vary in size from the aperture 500 to the outer edge of the bottom surface of the base 302. For example, stand-offs 551 adjacent the aperture 500 may be smaller or larger than stand-offs 551 adjacent the outer edge of the bottom surface of the base 302. The stand-offs 551 may be arranged in a uniform or non-uniform pattern. Non-uniform patterns may appear more organic. The stand-offs 551 may appear random, and may vary in size, such that the stand-offs 551 blend together to a greater or lesser degree.

The stand-offs 551 may be arranged in rows, rings, or bands extending at least partially around the aperture 500. For example, as illustrated in FIG. 6, the stand-offs 551 may be arranged in rings extending at least partially around the aperture 500. The stand-offs 551 in a first ring may align with the stand-offs 551 in a second ring. In some embodiments, the stand-offs 551 in the first ring may be radially offset from the stand-offs 551 in the second ring.

In some example embodiments, each of the stand-offs 551 may have a diameter ranging from about 1.0 mm to about 6.0 mm (e.g., about 1.5 mm to about 5.5 mm, about 2.0 mm to about 5.0 mm, about 2.5 mm to about 4.5 mm, or about 3.0 mm to about 4.0 mm).

The stand-offs 551 may extend to the outer edge 505 of the base 302 or may be spaced from the outer edge 505 of the base by about 0.5 mm to about 4.0 mm (e.g., about 1.0 mm to about 3.5. mm or about 1.5 mm to about 3.0 mm).

In the example of FIG. 6, the channels 605 are interconnected, and may also be non-linear. In some embodiments, the channels 605 may each have a width of about 0.01 mm to about 2.0 mm. The channels 605 may have a same or different width. The channels 605 adjacent the outer edge 505 of the bottom surface of the base 302 may have a smaller or a larger width than the channels 605 adjacent the aperture 700.

Referring to FIGS. 5 and 6, the conduit housing 362 of dressing interface 130 includes a recessed region 554 defining an entry surface 555. A primary port 660 and one or more ancillary ports 600 may be disposed on the entry surface 555. For example, the primary port 660 may be centrally located at an apex of the recessed region 554. The ancillary ports 600 may be positioned near diametrically opposing edges of the aperture 500 of the base 302 in some embodiments.

FIG. 7 is a cross-sectional view of the dressing interface 130 of FIG. 6 taken along line 7-7, illustrating additional details that may be associated with some embodiments. For example, a primary conduit 700 may fluidly couple the primary lumen interface 464 to the entry surface 555. In some embodiments, the primary conduit 700 may extend from the primary lumen interface 464 through the conduit housing 362, terminating at the primary port 660. In some embodiments, as shown in FIG. 7, the thickness of the base 302 may vary depending on the presence of the stand-offs 551 and the channels 605. For example, portions of the base 302 having the stand-offs 551 may be thicker than portions of the base 302 having the channels 605. In addition, some of the stand-offs 551 may have larger dimensions than others, such that the thickness of the base 302 is larger at portions of the base 302 in which larger stand-offs 551 are located. In some examples, the base 302 may have a thickness ranging from about 1.0 mm to about 3.0 mm (e.g., about 1.5 mm to about 2.5 mm).

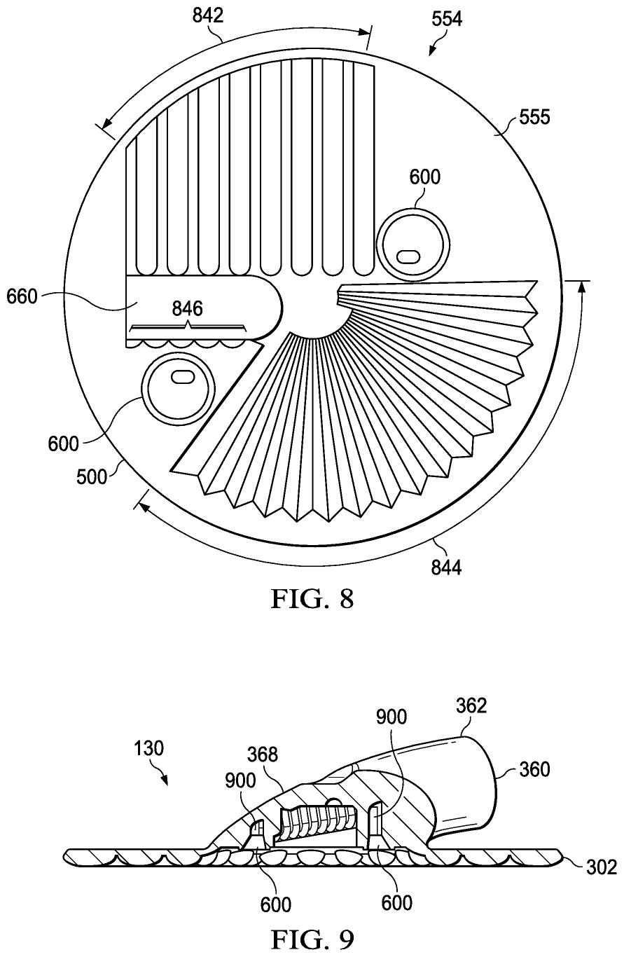

FIG. 8 is a detailed view of the recessed region 554 of FIG. 3, illustrating additional details that may be associated with some embodiments. The ancillary ports 600 may be positioned on the surface of the recessed region 554 as illustrated in the example of FIG. 8, with associated conduits extending internally from the ancillary ports 600 to ancillary lumen interfaces (hidden and not shown in this view).

As shown in FIG. 8, the primary port 660 may be centrally positioned within the recessed region 554 and extend from the central location to one side of recessed region 554. The ancillary ports 600 may be positioned on either side of the primary port 660 in some embodiments. In the example view of FIG. 8, the ancillary ports 800 are generally circular openings (each with raised circumferential edges) that extend toward a drainage point that opens into an internal conduit extending to the associated ancillary lumen interface (not shown). The openings of the conduits can be seen within the confines of the ancillary ports 600.

As shown in FIG. 8, the features in the recessed region 554 may be configured to direct liquid into the primary port 660. For example, structural serrated channels formed on various portions of the entry surface 555 of the recessed region 554 can direct liquid away from the ancillary ports 600 and to the primary port 660. A first channel section 842 can be positioned in association with an approximately half circle section of the recessed region 554 that is associated with one of the ancillary ports 600. As illustrated in the example of FIG. 8, the first channel section 842 may comprise linear serrated channels. The material that comprises the ceiling of this section of recessed region 554 can cover and contain the conduit that extends between the ancillary port 600 and its interface (not shown). This ceiling or wall may have an array of serrated channels or striations configured to direct liquid towards the primary port 660 at the center of the recessed region 554.

A second channel section 844 may be constructed in an approximately one-third circular section of the recessed region 554. As illustrated in FIG. 8, the second channel section 844 may comprise radial serrated channels. In some embodiments, the channels in the second channel section 844 may extend directly to the primary port 660. These radial serrated channels can be directed from a perimeter of recessed region 554 towards the apex of the recessed region 554 that drains into the primary port 660. The second channel section 844 can span the recessed region 554 from one of the ancillary ports 600 radially around approximately one-third of the circle to another of the ancillary ports 600. The radial channels of the second channel section 844 can be configured to direct liquid centrally to the primary port 660, rather than being conducted to either of the ancillary ports 600.

In some embodiments, one of the ancillary ports 600 may overhang the primary port 660, as illustrated in the example of FIG. 8, with a wall section supporting it. The wall section may also have serrated or striated channels 846 that extend from the opening of the ancillary port 600 towards the opening of primary port 660.

FIG. 9 is a cross-sectional view of the dressing interface 130 of FIG. 6, illustrating additional details that may be associated with some embodiments.

As illustrated in the example of FIG. 9, in some embodiments ancillary conduits can fluidly couple the ancillary lumen interfaces 448 (see FIG. 4) to the entry surface 555. For example, the ancillary conduits 902 can extend internally through the conduit housing 362 from the ancillary ports 600 to the ancillary lumen interfaces 448.

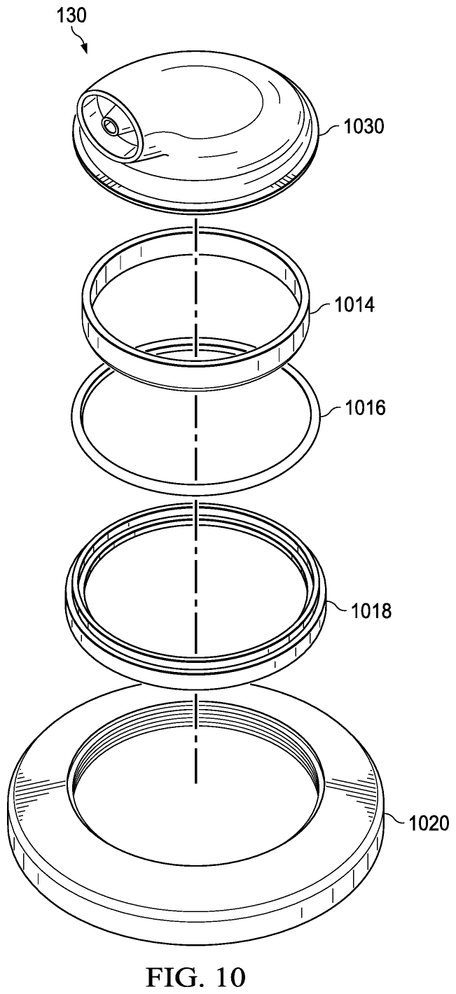

FIG. 10 is an exploded view of the dressing interface 130, illustrating additional details that may be associated with some embodiments.

In some embodiments, the dressing interface 130 employs a hard plastic inner core that forms a bearing surface to enable a rubber o-ring to seal against it and also to enable the bearing surface to slide past with relatively low friction. Bonded to the hard plastic inner core is a soft thermoplastic or elastomeric polymer that acts as a protective and cushioning cover. FIG. 9 shows the various circular ring components that go together to make up the swivel connection. A top rotating PVC component 1030 covers a top ABS insert ring 1014 which itself is surrounded by a rubber o-ring 1016. A bottom ABS insert ring 1018 is shown that holds an o-ring 1016 captive between the bottom ABS insert ring 1018 and the top ABS insert ring 1014. Each of these rings is then fitted within a bottom PVC ring 1020 which comes into contact with the base of the dressing interface and/or with the dressing 104 itself.

The internal features and elements associated with the dressing interface 130 described above in conjunction with the example embodiment of FIG. 3 may be equally applicable to the example of FIG. 10, and may be integrated into the inside structure of top rotating PVC component 1030 by direct molding of the component or by positioning a molded insert into a shell to form the top rotating PVC component 1030. In any event, the same benefits of the liquid-preferencing structures surrounding the fluid pathway ports described above may be obtainable with the rotating functionality of the alternate embodiment described.

In operation, the tissue interface 114 may be placed within, over, on, or otherwise proximate to a tissue site. The cover 116 may be placed over the tissue interface 114 and sealed to an attachment surface near the tissue site. For example, the cover 116 may be sealed to undamaged epidermis peripheral to a tissue site. Thus, the dressing 104 can provide a sealed therapeutic environment proximate to a tissue site, substantially isolated from the external environment. The dressing interface 130 may be positioned on the dressing 104, such that the dressing interface 130 does not directly contact a tissue site, but instead may contact only the dressing 104. For example, in some embodiments the base 302 may be directly adhered to the tissue interface 114 or may be positioned and adhered using the cover 116 of the dressing 104. The dressing interface 130 may be positioned so that the aperture 500 of the base 302 is in direct contact with the tissue interface 114. In other embodiments, the dressing 104 may be omitted and the dressing interface 130 may contact a tissue site directly.

The negative-pressure source 102 can reduce the pressure in the sealed therapeutic environment. Negative pressure applied across the tissue site through the tissue interface 114 in the sealed therapeutic environment can induce macrostrain and micro-strain in the tissue site, as well as remove exudates and other fluids from the tissue site. For example, exudate may be drawn from the tissue site, flow through the aperture 500 and the primary conduit, and through the tubing sections 170 into the container 106.

The channels 605 between the stand-offs 551 may be in fluid communication with the aperture 500, such that the channels 605 are configured to direct liquid into aperture 500 and the recessed region 554. The various internal features and elements of the recessed region 554 can be structured to draw liquid from most points within the recessed region 554 towards the primary port 660. The routing of liquids into the primary port 660 can maintain the ancillary ports 600 open for pressure measurement purposes.

The placement of the ancillary ports 600 near the perimeter of the recessed region 554 at a level that is close to the surface of the tissue interface 114 when the dressing interface 130 is positioned thereon can also assist in directing liquid into the primary port 660. In other words, if the dressing interface 130 is positioned on the dressing 104, the ancillary ports 600 are in contact, or are nearly in contact, with the surface of the tissue interface 114. In this manner, the likelihood of splashed or agitated liquid being directed into the ancillary ports 600 can be minimized.

The systems, apparatuses, and methods described herein may provide significant advantages. For example, the stand-offs 551 may provide a soft interface area where fluid is removed. In some embodiments, for example, the inclusion of the stand-offs 551 on the bottom surface of the base 302 can reduce and/or substantially prevent tissue damage when used over tissue without a dressing 104. For example, the stand-offs 551 may be consist of non-linear features to minimize pressure points and strain on tissue.

In addition, the bottom surface of the base 302 may appear gentler. In some embodiments, for example, the stand-offs 551 may also be arranged in a mixture of patterns or textures, or random-like patterns. The patterns can mimic organic shapes in some examples. Moreover, the pattern may be varied between dressing interfaces to vary pressure points if a dressing interface is changed during treatment.

The stand-offs 551 can also function as a manifold. For example, the stand-offs 551 may define channels on the interface area, which may be interconnected to provide redundant fluid pathways to the central area of the dressing interface 130, and can manifold pressures and fluids well even if the dressing interface 130 is placed off-center over the dressing 104.

Flow pathways may extend through the dressing interface 130 to the primary port 660. The elbow region 368 can redirect fluid flow from the dressing 104 positioned beneath the dressing interface 130 to the primary lumen interface 364 in a manner that allows the therapy system 100 to be placed on the dressing 104 and be maintained close to the dressing surface.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use. For example, in some configurations the dressing 104, the container 106, or both may be eliminated or separated from other components for manufacture or sale. In other example configurations, the controller 108 may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

1. A dressing interface for a negative-pressure treatment system, the dressing interface comprising:
a housing comprising an entry surface having first channels;
a primary conduit through the housing and terminating on the entry surface;
an ancillary conduit through the housing and terminating on the entry surface; and
a base coupled to the housing, the base comprising:
an aperture, and
a plurality of stand-offs having rounded surfaces defining second channels configured to facilitate flow of liquid to the first channels through the aperture.

2. The dressing interface of claim 1, wherein the base is formed of polyvinyl chloride.

3. The dressing interface of claim 1, wherein the plurality of stand-offs have a hardness of about 60 Shore A.

4. The dressing interface of claim 1, wherein each of the plurality of stand-offs has a generally round shape.

5. The dressing interface of claim 1, wherein the first channels are adapted to direct liquid away from the ancillary conduit.

6. The dressing interface of claim 1, wherein stand-offs adjacent the aperture of the base have smaller dimensions than stand-offs adjacent an edge of the base.

7. The dressing interface of claim 1, wherein the plurality of stand-offs are distributed in an irregular pattern on the base.

8. The dressing interface of claim 1, wherein the plurality of stand-offs are distributed in a regular pattern on the base.

9. The dressing interface of claim 1, wherein each of the plurality of stand-offs has a same size.

10. The dressing interface of claim 1, wherein the base has a thickness ranging from about 1.0 mm to about 3.0 mm.

11. The dressing interface of claim 1, wherein each of the stand-offs has a diameter ranging from about 1.0 mm to about 6.0 mm.

12. The dressing interface of claim 1, wherein each of the second channels is interconnected with other ones of the second channels.

13. The dressing interface of claim 1, wherein each of the second channels has a width of about 0.01 mm to about 2.0 mm.

14. A negative-pressure treatment system comprising:
a conduit comprising a primary lumen and a secondary lumen;
a negative-pressure source coupled to the primary lumen; and
a dressing interface coupled to the conduit, the dressing interface comprising:
a base and a housing,
first channels associated with the housing and configured to preference fluid into the primary lumen,
an aperture in the base, and
a plurality of stand-offs having rounded edges defining second channels configured to facilitate flow of fluid to the first channels through the aperture.

15. The negative-pressure treatment system of claim 14, wherein the base is formed of polyvinyl chloride and the plurality of stand-offs have a hardness of about 60 Shore A.

16. The negative-pressure treatment system of claim 14, wherein each of the plurality of stand-offs has a generally round shape.

17. The negative-pressure treatment system of claim 14, wherein stand-offs adjacent the aperture in the base have smaller dimensions than stand-offs adjacent an outer edge of the base.

18. The negative-pressure treatment system of claim 14, wherein the base has a thickness ranging from about 1.0 mm to about 3.0 mm.

19. The negative-pressure treatment system of claim 14, wherein each of the stand-offs has a diameter ranging from about 1.0 mm to about 6.0 mm, and each of the second channels is interconnected with other ones of the second channels.

20.-29. (canceled)
